(19) United States
(12) Patent Application Publication
(10) Pub. No.: US 2025/0010040 A1
(43) Pub. Date: Jan. 9, 2025

(54) CATHETER ASSEMBLY AND BLOOD VESSEL PUNCTURE SYSTEM

(71) Applicant: TERUMO KABUSHIKI KAISHA, Tokyo (JP)

(72) Inventor: Shinichi MIZUNO, Nakakoma-gun (JP)

(73) Assignee: TERUMO KABUSHIKI KAISHA, Tokyo (JP)

(21) Appl. No.: 18/890,209

(22) Filed: Sep. 19, 2024

Related U.S. Application Data
(63) Continuation of application No. PCT/JP2023/008210, filed on Mar. 6, 2023.

(30) Foreign Application Priority Data
Mar. 30, 2022 (JP) 2022-055585

(51) Int. Cl. A61M 25/06

(52) U.S. Cl. CPC A61M 25/0606; A61M 2205/0227; A61M 2205/3306; A61M 2205/502; A61M 2205/583

(57) ABSTRACT

A catheter assembly includes: a catheter member including a hollow catheter shaft; a needle member including a hollow needle body located in a lumen of the catheter shaft; and a guide wire located in a lumen of the needle body. A distal end opening communicating with the lumen of the needle body is formed on a blade surface of a distal end of the needle body. A light emission unit configured to emit near-infrared light is located on a distal end of the guide wire. In a plan view in which the blade surface is viewed in a direction orthogonal to an axis of the needle body in an initial state, the light emission unit overlaps the distal end opening.

### CROSS-REFERENCE TO RELATED APPLICATIONS

The present application is a bypass continuation of PCT Application No. PCT/JP2023/008210, filed on Mar. 6, 2023, which claims priority to Japanese Patent Application No. JP2022-055585, filed on Mar. 30, 2022. The entire contents of these applications are incorporated by reference herein.

### BACKGROUND

The present disclosure relates to a catheter assembly and a blood vessel puncture system.

For example, JP 10-99443 A discloses a catheter assembly provided with a hollow catheter shaft, a hollow needle body inserted into a lumen of the catheter shaft, and a guide wire inserted into the lumen of the needle body. A distal end opening communicating with the lumen of the needle body is formed on a blade surface on the distal end of the needle body.

### SUMMARY

In general, in a catheter assembly, it is confirmed that a blade surface of a needle body enters a blood vessel by visually observing a backflow (flashback) of blood via a lumen of the needle body. In this case, because the flashback can be confirmed after an elapse of a predetermined time after the blade surface of the needle body enters the blood vessel, it is not possible to immediately recognize that the blade surface of the needle body enters the blood vessel.

An object of the present disclosure is to solve the above-described problems.

According to one aspect of the present disclosure, a catheter assembly includes a catheter member including a hollow catheter shaft, a needle member including a hollow needle body inserted into a lumen of the catheter shaft, and a guide wire inserted into a lumen of the needle body, in which a distal end opening communicating with the lumen of the needle body is formed on a blade surface of a distal end of the needle body, a light emission unit that emits near-infrared light is provided on a distal end of the guide wire, and the light emission unit is located so as to overlap the distal end opening in a plan view in which the blade surface is seen in a direction orthogonal to an axis of the needle body in an initial state.

According to another aspect of the present disclosure, a blood vessel puncture system includes the catheter assembly described above, an irradiation unit that irradiates a distal end of the catheter assembly with which a living body site is punctured with light in a near-infrared region, a light reception unit that receives reflected light reflected by the living body site and the catheter assembly out of the light and the near-infrared light emitted by the light emission unit to create a light reception image, and an image display unit that displays the light reception image.

According to certain aspects, in the initial state of the catheter assembly, the light emission unit is located at the position overlapping the distal end opening when the blade surface is seen in the direction orthogonal to the axis of the needle body. In this case, because the near-infrared light emitted by the light emission unit is guided to the outside of the needle body via the distal end opening, this can be visualized by the light reception image created on the basis of the near-infrared light. The near-infrared light is absorbed by hemoglobin in the blood flowing in the blood vessel. Therefore, the appearance of the light emission unit in the light reception image changes before and after the blade surface enters the blood vessel. Therefore, the user can immediately recognize that the blade surface enters the blood vessel.

### BRIEF DESCRIPTION OF DRAWINGS

FIG. 1 is a schematic configuration diagram of a blood vessel puncture system according to an embodiment of the present invention;

FIG. 2 is an exploded perspective view of a catheter assembly in FIG. 1;

FIG. 3A is a longitudinal cross-sectional view of a distal end of the catheter assembly in FIG. 1;

FIG. 3B is a plan view of a blade surface as seen in a direction orthogonal to an axis of a needle body in the catheter assembly in FIG. 1;

FIG. 4 is a first illustrative diagram of a blood vessel puncture procedure of the catheter assembly in FIG. 1;

FIG. 5 is a light reception image in a state of FIG. 4;

FIG. 6 is a second illustrative diagram of a blood vessel puncture procedure of the catheter assembly in FIG. 1;

FIG. 7 is a light reception image in a state of FIG. 6;

FIG. 8 is a third illustrative diagram of a blood vessel puncture procedure of the catheter assembly in FIG. 1; and

FIG. 9 is a schematic configuration diagram of a catheter assembly according to a modified example.

### DETAILED DESCRIPTION

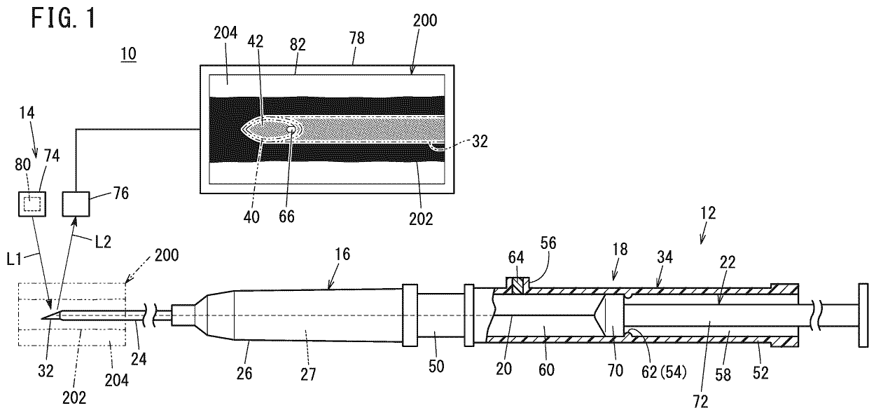

As illustrated in FIG. 1, a blood vessel puncture system 10 according to an embodiment of the present invention is provided with a catheter assembly 12 and a visualization device 14. The catheter assembly 12 is configured as, for example, an indwelling needle for administering an infusion solution (medical solution) into a blood vessel 202 of a living body site 200. Note that, the catheter assembly 12 is not limited to the indwelling needle for administering the infusion solution.

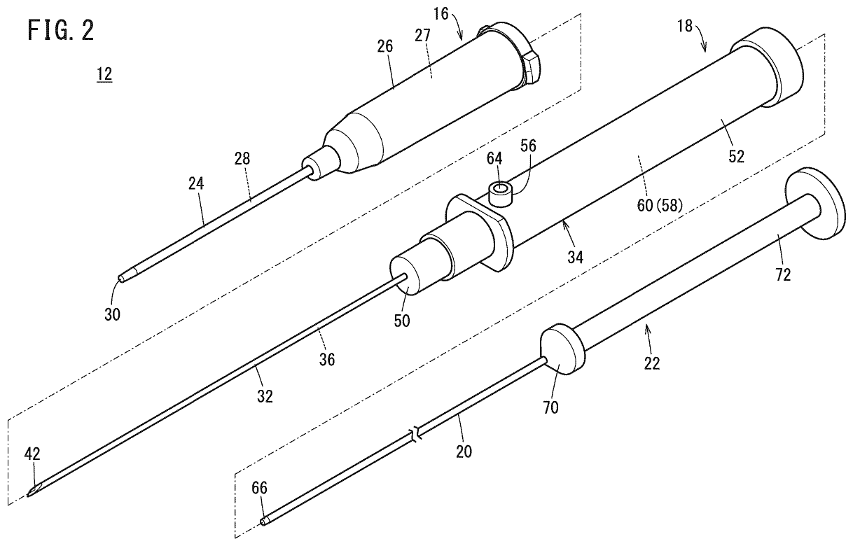

As illustrated in FIGS. 1 and 2, the catheter assembly 12 is provided with a catheter member 16, a needle member 18, a guide wire 20, and a wire operation unit 22. The catheter member 16 includes a hollow catheter shaft 24 and a catheter hub 26.

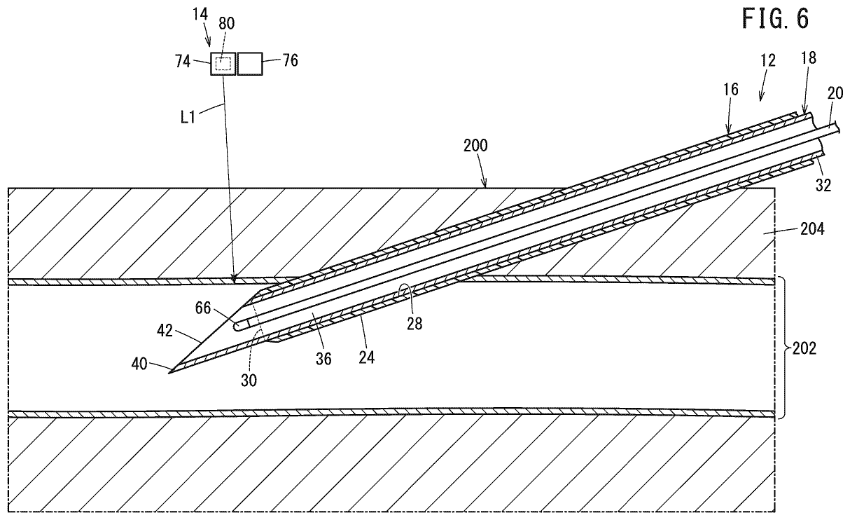

In FIG. 2, the catheter shaft 24 is flexible. The catheter shaft 24 is a tubular member persistently insertable into the blood vessel 202 (vein or artery) of the living body site 200 (refer to FIG. 6). The catheter shaft 24 includes a lumen 28 extending in an axial direction over an entire length thereof. The lumen 28 of the catheter shaft 24 communicates with an opening 30 on a distal end of the catheter shaft 24.

A component material of the catheter shaft 24 is not especially limited, but a transparent or translucent resin material, especially a soft resin material is suitable; there are for example, a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, a mixture of the olefin-based resin and an ethylene-vinyl acetate copolymer and the like.

The catheter hub 26 is provided on a proximal end of the catheter shaft 24. The catheter hub 26 is formed into a cylindrical shape. The catheter hub 26 is preferably formed of a material harder than that of the catheter shaft 24. A component material of the catheter hub 26 is not especially limited, but a thermoplastic resin such as polypropylene, polycarbonate, polyamide, polysulfone, polyarylate, a methacrylate-butylene-styrene copolymer, polyurethane, an acrylic resin, and an ABS resin, for example, can be suitably applied.

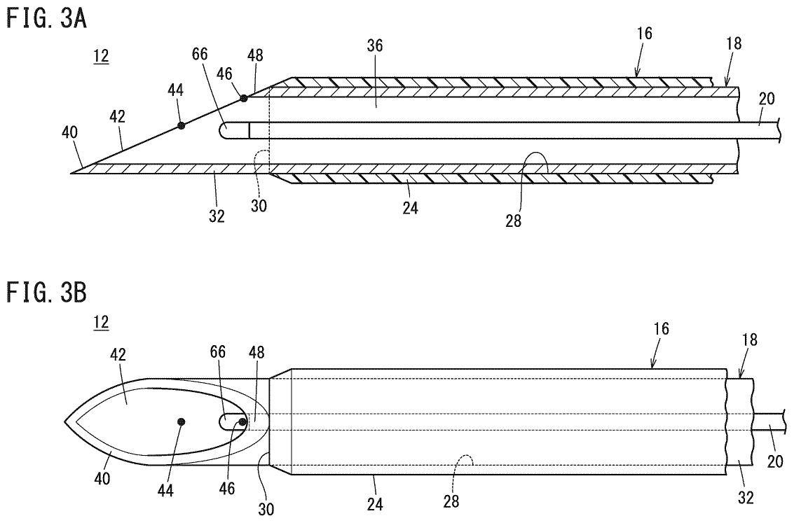

The needle member 18 is provided with a hollow needle body 32 and a needle hub 34. The needle body 32 is a rigid tubular member capable of puncturing the living body site 200 (refer to FIG. 6). The needle body 32 includes a lumen 36 extending in an axial direction over an entire length thereof. The needle body 32 is inserted into the lumen 28 of the catheter shaft 24 and a lumen 27 of the catheter hub 26 in an initial state (assembled state) of the catheter assembly 12 (refer to FIGS. 1 and 3A).

A component material of the needle body 32 includes, for example, a metal material such as stainless steel, aluminum, an aluminum alloy, titanium, and a titanium alloy. The needle body 32 does not transmit but reflects light L1 from the visualization device 14 (refer to FIG. 1). The needle body 32 is formed to be sufficiently longer than the catheter shaft 24.

As illustrated in FIGS. 3A and 3B, the needle body 32 protrudes from the opening 30 of the catheter shaft 24 in the initial state of the catheter assembly 12. A distal end of the needle body 32 includes a blade surface 40 inclined with respect to an axis of the needle body 32. A distal end opening 42 communicating with the lumen 36 of the needle body 32 is formed on the blade surface 40.

The distal end opening 42 includes an opening center portion 44 and an opening proximal end 46. The opening center portion 44 is located at the center portion in the axial direction of the needle body 32 of the distal end opening 42. The opening proximal end 46 is located on a proximal end in the axial direction of the needle body 32 of the distal end opening 42. In other words, the opening proximal end 46 is adjacent to a jaw 48 of the blade surface 40 in a distal end direction.

In FIGS. 1 and 2, the needle hub 34 includes a needle fixing unit 50, a cylindrical portion 52, a movement restriction unit 54, and a port 56. The needle fixing unit 50 forms a distal end of the needle hub 34. A proximal end of the needle body 32 is fixed to the needle fixing unit 50. The cylindrical portion 52 extends in an extending direction of the needle body 32. A distal end of the cylindrical portion 52 is continuous to the needle fixing unit 50. The cylindrical portion 52 includes a lumen 58 communicating with the lumen 36 of the needle body 32 and opened to a proximal end of the needle hub 34.

As illustrated in FIG. 1, a piston 70 (to be described below) of the wire operation unit 22 is inserted into the lumen 58 of the cylindrical portion 52. A space in the distal end direction of the piston 70 in the lumen 58 of the cylindrical portion 52 is a chamber 60 for flashback in which blood flows backward when the blade surface 40 of the needle body 32 enters the blood vessel 202 (refer to FIG. 6). The cylindrical portion 52 functions as an operation unit of the catheter assembly 12.

The movement restriction unit 54 restricts movement of the piston 70 in a proximal end direction. The movement restriction unit 54 is two protrusions 62 protruding radially inward from an inner peripheral surface of the cylindrical portion 52. The two protrusions 62 are located so as to face each other. The protrusion 62 is formed to have a semicircular longitudinal cross section in an axial direction of the cylindrical portion 52. The position, size, and shape of the protrusion 62 may be designed appropriately. The movement restriction unit 54 may be one or three or more protrusions 62. The movement restriction unit 54 may be one protrusion 62 extending in an annular shape on the inner peripheral surface of the cylindrical portion 52. The movement restriction unit 54 may have any appropriate configuration as long as the movement of the piston 70 in the proximal end direction can be restricted.

In FIGS. 1 and 2, the port 56 is provided on the distal end of the cylindrical portion 52. The port 56 is provided with a filter 64. The filter 64 allows air present in the chamber 60 to flow to the outside of the cylindrical portion 52 and blocks an inflow of the air from the outside of the cylindrical portion 52 into the chamber 60. The filter 64 prevents circulation of the blood.

The guide wire 20 extends in the distal end direction from a distal end of the piston 70. The guide wire 20 is a member for guiding the catheter shaft 24 into the blood vessel 202. A core material of the guide wire 20 is formed of, for example, a superelastic alloy. A surface of the guide wire 20 is coated with a hydrophilic polymer. A distal end of the guide wire 20 has a flexible structure (for example, a coil and the like) to increase safety to the blood vessel 202. The distal end of the guide wire 20 is formed into a round shape (for example, into a hemispherical shape).

As illustrated in FIGS. 3A and 3B, a light emission unit 66 that emits near-infrared light L2 is provided on the distal end of the guide wire 20. The near-infrared light L2 emitted by the light emission unit 66 is received by the visualization device 14 and displayed on a light reception image 82 (refer to FIG. 1). A length of the light emission unit 66 in an extending direction of the guide wire 20 is set to, for example, 0.3 mm or longer and 0.5 mm or shorter. When the length of the light emission unit 66 is 0.3 mm or longer, the light emission unit 66 is easily visually recognized in the light reception image 82. Note that, the length of the light emission unit 66 can be appropriately set.

In the initial state of the catheter assembly 12, the light emission unit 66 is located at a position overlapping the distal end opening 42 of the needle body 32 in a plan view (in a plan view of FIG. 3B) in which the blade surface 40 is seen in a direction orthogonal to the axis of the needle body 32. Specifically, in the initial state of the catheter assembly 12, the light emission unit 66 is located in the proximal end direction from the opening center portion 44 of the distal end opening 42. In other words, the light emission unit 66 is located at a position overlapping the opening proximal end 46 of the distal end opening 42 in a plan view of FIG. 3B. In this case, it is possible to easily recognize whether an entire blade surface 40 is inserted into the blood vessel 202 from the light reception image 82.

In the present embodiment, a distal end of the light emission unit 66 is located in the proximal end direction from the opening center portion 44 of the distal end opening 42 in the initial state of the catheter assembly 12. An entire light emission unit 66 is located in the lumen 36 of the needle body 32. That is, the light emission unit 66 does not protrude to the outside of the needle body 32 from the distal end opening 42. As a result, it is possible to suppress an increase in puncture resistance by the light emission unit 66.

The size, shape, and position of the light emission unit 66 can be designed appropriately. The light emission unit 66 may extend so as to overlap a range from the opening center portion 44 to the opening proximal end 46 of the distal end opening 42 in the axial direction of the needle body 32 in a plan view of FIG. 3B. In the initial state, the light emission unit 66 may be located in the proximal end direction from the opening center portion 44 of the distal end opening 42. The light emission unit 66 is preferably located so as to overlap at least the opening proximal end 46 of the distal end opening 42 in a plan view of FIG. 3B. Note that, the light emission unit 66 may be located so as not to overlap the opening proximal end 46 of the distal end opening 42 in a plan view of FIG. 3B.

A peak wavelength of the near-infrared light L2 emitted by the light emission unit 66 is in a range of wavelengths that is easily absorbed by hemoglobin in the blood and easily passes through a skin 204. In general, the skin 204 easily transmits a wavelength of 700 nm or longer and 1000 nm or shorter. Reduced hemoglobin in the blood flowing through the vein easily absorbs light having a wavelength around 660 nm. Furthermore, oxygenated hemoglobin in the blood flowing through the vein easily absorbs light having a wavelength around 940 nm.

Therefore, the peak wavelength of the near-infrared light L2 emitted by the light emission unit 66 is preferably in a range of 700 nm or longer and 1000 nm or shorter. In a case of the catheter assembly 12 (catheter for venous insertion) used for venous puncture, the peak wavelength of the near-infrared light L2 emitted by the light emission unit 66 is preferably in a range of 700 nm or longer and 800 nm or shorter, and more preferably in a range of 700 nm or longer and 750 nm or shorter. Furthermore, in a case of the catheter assembly 12 (catheter for arterial insertion) used for arterial puncture, the peak wavelength of the near-infrared light L2 emitted by the light emission unit 66 is preferably in a range of 800 nm or longer and 1000 nm or shorter, more preferably in a range of 850 nm or longer and 950 nm or shorter, and still more preferably around 940 nm.

The light emission unit 66 is formed by applying a light emitting material to the distal end of the guide wire 20. Such light emission unit 66 can be easily obtained, for example, by immersing the distal end of the guide wire 20 in a liquid light emitting material. As the light emitting material, a fluorescent material (near-infrared fluorescent dye) that emits the near-infrared light L2 when being irradiated with the light L1 in a near-infrared region emitted by the visualization device 14 is used. A thickness of the fluorescent material is set to, for example, about 3 μm. Examples of such fluorescent material include those disclosed in JP 2014-136115 A, for example.

As the light emitting material, a phosphorescent material that emits the near-infrared light L2 may be used. In a case of using such phosphorescent material, by irradiating the light emission unit 66 with light in advance (before performing a procedure of blood vessel puncture using the blood vessel puncture system 10), the light emission unit 66 can be made to be able to emit light.

As illustrated in FIGS. 1 and 2, the wire operation unit 22 is a member for operating the guide wire 20 in an axial direction. The wire operation unit 22 includes a piston 70 and a plunger 72. The piston 70 is arranged in the lumen 58 of the cylindrical portion 52. The piston 70 slides in the lumen 58 of the cylindrical portion 52 in the axial direction of the cylindrical portion 52. A distal end of the plunger 72 is attached to a proximal end surface of the piston 70. A proximal end of the plunger 72 protrudes from a proximal end of the cylindrical portion 52.

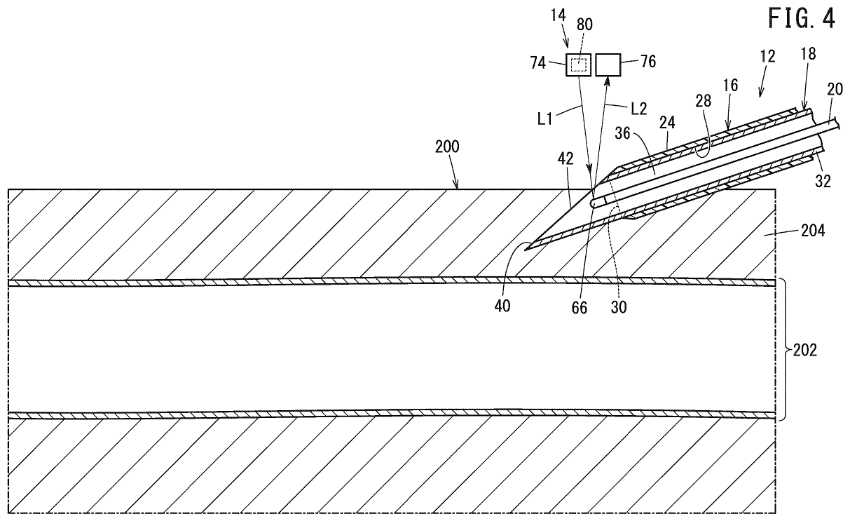

In FIG. 1, the visualization device 14 is provided with an irradiation unit 74, a light reception unit 76, and an image display unit 78. The irradiation unit 74 and the light reception unit 76 are located above the living body site 200 (in a direction in which the distal end opening 42 of the needle body 32 faces) (refer to FIG. 4). The irradiation unit 74 irradiates the living body site 200 (visualization target object) punctured with the catheter assembly 12 with the light L1. The irradiation unit 74 includes a light source 80 that emits the light L1 in the near-infrared region.

For example, the light L1 emitted by the light source 80 preferably has a wavelength in a range of 700 nm or longer and 2500 nm or shorter, more preferably has a wavelength in a range of 700 nm or longer and 1400 nm or shorter, and still more preferably has a wavelength in a range of 780 nm or longer and 1050 nm or shorter. Such near-infrared light L2 is absorbed by hemoglobin in the blood.

The light reception unit 76 is a camera (imaging unit) that receives reflected light reflected by the living body site 200, the needle body 32 and the like out of the light L1 emitted by the light source 80 and the near-infrared light L2 emitted by the light emission unit 66. For example, a CCD camera and the like is used as the light reception unit 76. The light reception unit 76 creates the light reception image 82 on the basis of the reflected light and the near-infrared light L2. The image display unit 78 displays the light reception image 82.

Next, the procedure of blood vessel puncture using the blood vessel puncture system 10 is described. In the initial state of the catheter assembly 12, the blade surface 40 protrudes in the distal end direction from the opening 30 of the catheter shaft 24 in a state of facing upward.

First, a user sets the visualization device 14. Specifically, as illustrated in FIG. 4, the irradiation unit 74 and the light reception unit 76 are arranged above the living body site 200 (for example, the forearm of the human body) to be punctured. Then, the living body site 200 is irradiated with the light L1 by the irradiation unit 74, and the living body site 200 is punctured with the distal end of the catheter assembly 12. Note that, in FIG. 4, the blade surface 40 does not enter the blood vessel 202.

Then, the light L1 emitted by the irradiation unit 74 is reflected by the living body site 200 and the needle body 32 and applied to the light emission unit 66. Note that, the light L1 in the near-infrared region is absorbed by hemoglobin in the blood flowing in the blood vessel 202 of the living body site 200. When being irradiated with the light L1, the light emission unit 66 emits the near-infrared light L2. The light reception unit 76 receives the light (reflected light) reflected by the living body site 200 and the needle body 32 out of the light L1 and the near-infrared light L2 emitted by the light emission unit 66 to create the light reception image 82. The light reception image 82 created by the light reception unit 76 is displayed on the image display unit 78.

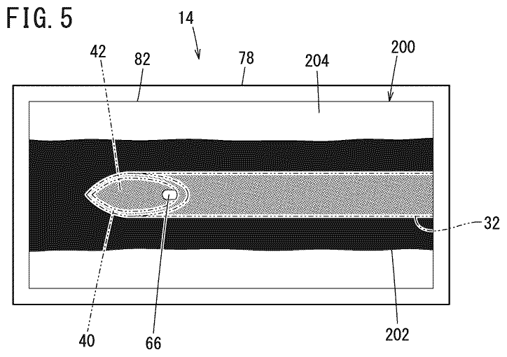

As illustrated in FIG. 5, the skin 204, the blood vessel 202, the needle body 32, and the light emission unit 66 are displayed on the light reception image 82. Specifically, in the light reception image 82, the skin 204 and the light emission unit 66 are displayed brighter than the needle body 32.

Subsequently, as illustrated in FIG. 6, when the catheter assembly 12 is pushed forward, the blade surface 40 of the needle body 32 is inserted into the blood vessel 202, and the light emission unit 66 is located in the blood vessel 202. Then, the light L1 is absorbed by hemoglobin in the blood flowing in the blood vessel 202, so that the light emission unit 66 is not irradiated with the same.

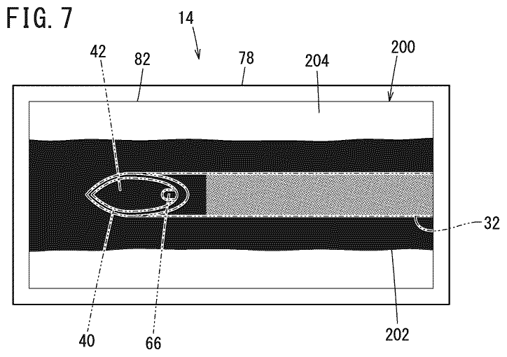

Therefore, as illustrated in FIG. 7, an appearance of the light emission unit 66 changes (the light emission unit 66 becomes dark) in the light reception image 82. In other words, the brightness of the light emission unit 66 becomes substantially the same as the brightness of the blood vessel 202. Therefore, the user can immediately recognize that the blade surface 40 enters the blood vessel 202 based on the light reception image 82. In other words, the user can recognize that the entire blade surface 40 is inserted into the blood vessel 202 based on the light reception image 82.

Note that, when the blade surface 40 is inserted into the blood vessel 202, the blood in the blood vessel 202 is guided from the distal end opening 42 of the needle body 32 to the chamber 60 via the lumen 36. As a result, the user can reconfirm that the blade surface 40 is inserted into the blood vessel 202 by flashback.

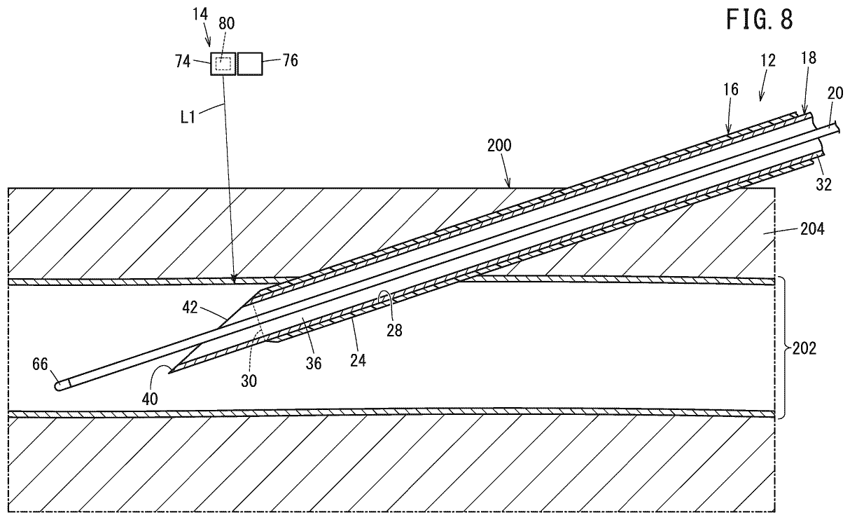

Thereafter, as illustrated in FIG. 8, the user moves the wire operation unit 22 in the distal end direction with respect to the needle hub 34 to cause the guide wire 20 to protrude from the distal end opening 42 of the needle body 32. Then, the catheter shaft 24 is inserted into the blood vessel 202 along the guide wire 20. As a result, the catheter shaft 24 is indwelled in the blood vessel 202. Subsequently, the infusion solution is administered into the blood vessel 202 via the catheter member 16.

The present embodiment has the following effects.

According to the present embodiment, in the initial state of the catheter assembly 12, the light emission unit 66 is located at the position overlapping the distal end opening 42 when the blade surface 40 is seen in the direction orthogonal to the axis of the needle body 32. In this case, because the near-infrared light L2 emitted by the light emission unit 66 is guided to the outside of the needle body 32 via the distal end opening 42, this can be visualized by the light reception image 82 created on the basis of the near-infrared light L2. The near-infrared light L2 is absorbed by hemoglobin in the blood flowing in the blood vessel 202. Therefore, the appearance of the light emission unit 66 in the light reception image 82 changes before and after the blade surface 40 enters the blood vessel 202. Therefore, the user can immediately recognize that the blade surface 40 enters the blood vessel 202.

The needle member 18 includes the needle hub 34 provided on the proximal end of the needle body 32. The needle hub 34 includes the movement restriction unit 54 that restricts the movement of the guide wire 20 in the proximal end direction with respect to the needle body 32 in the initial state of the catheter assembly 12.

According to such configuration, it is possible to prevent the distal end of the light emission unit 66 from being displaced in the proximal end direction from the proximal end of the distal end opening 42 in the initial state of the catheter assembly 12.

The distal end opening 42 includes the opening center portion 44 located at the center portion in the axial direction of the needle body 32. In the initial state of the catheter assembly 12, the light emission unit 66 is located in the proximal end direction from the opening center portion 44.

According to such configuration, it is possible to accurately recognize that the entire blade surface 40 is inserted into the blood vessel 202.

The distal end opening 42 includes the opening proximal end 46 located on the proximal end in the axial direction of the needle body 32. In the initial state of the catheter assembly 12, the light emission unit 66 is located so as to overlap the opening proximal end 46 in a plan view.

According to such configuration, it is possible to more accurately recognize that the entire blade surface 40 is inserted into the blood vessel 202.

The light emission unit 66 includes the fluorescent material that emits the near-infrared light L2 when being irradiated with the light L1.

According to such configuration, the configuration of the light emission unit 66 can be simplified.

The blood vessel puncture system 10 is provided with the catheter assembly 12, the irradiation unit 74, the light reception unit 76, and the image display unit 78. The irradiation unit 74 irradiates the distal end of the catheter assembly 12 with which the living body site 200 is punctured with the light L1 of the near-infrared region. The light reception unit 76 receives the reflected light reflected by the catheter assembly 12 out of the light L1 and the near-infrared light L2 emitted by the light emission unit 66 to create the light reception image 82. The image display unit 78 displays the light reception image 82.

According to such configuration, it is possible to efficiently perform the procedure of the blood vessel puncture while viewing the light reception image 82.

### Modified Example

Next, a catheter assembly 12a according to a modified example is described. Note that, in the present modified example, the same configuration as that of the above-described catheter assembly 12 is denoted by the same reference numeral, and a detailed description thereof is omitted.

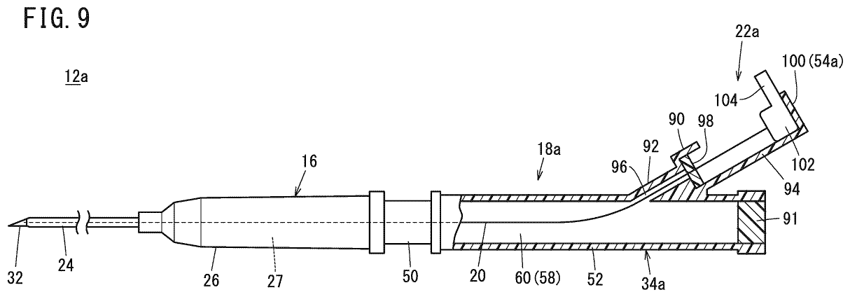

As illustrated in FIG. 9, the catheter assembly 12a is provided with a catheter member 16, a needle member 18a, a guide wire 20, and a wire operation unit 22a. The needle member 18a includes a needle body 32 and a needle hub 34a. The needle hub 34a includes a needle fixing unit 50, a cylindrical portion 52, a wire introduction unit 90, and a movement restriction unit 54a.

The cylindrical portion 52 is not provided with the port 56 described above. A lumen 58 of the cylindrical portion 52 is a chamber 60 for flashback in which blood flows backward when a blade surface 40 of the needle body 32 enters a blood vessel 202. A proximal end of the cylindrical portion 52 is provided with a filter 91. The filter 91 is configured similarly to the filter 64 described above.

The wire introduction unit 90 includes a connector 92 and a supporter 94. The connector 92 is provided on the cylindrical portion 52. The connector 92 includes a lumen 96 communicating with the lumen 58 of the cylindrical portion 52. The connector 92 is provided with a slit valve 98 through which the guide wire 20 is inserted.

The slit valve 98 prevents the blood flowing backward into the chamber 60 from leaking from the lumen 96 of the wire introduction unit 90 to the outside. The supporter 94 extends so as to be inclined radially outward from the connector 92 in a proximal end direction. The movement restriction unit 54a includes a restriction wall 100 provided on an extending end of the supporter 94. The restriction wall 100 restricts the movement of the wire operation unit 22a in a direction away from the slit valve 98.

The wire operation unit 22a includes a base 102 and a handle 104. The base 102 is attached to a proximal end of the guide wire 20. The base 102 is in contact with the restriction wall 100 in the initial state of the catheter assembly 12a. The handle 104 protrudes from the base 102. The handle 104 has the size and shape that allow a user to easily grip with fingers.

In the catheter assembly 12a according to the present modified example, the configuration similar to that of the above-described catheter assembly 12 has a similar effect. According to the present modified example, because the base 102 is in contact with the restriction wall 100 in the initial state of the catheter assembly 12a, the guide wire 20 does not retract with respect to the needle member 18a. Therefore, it is possible to prevent a distal end of a light emission unit 66 from being displaced in a proximal end direction from a proximal end of a distal end opening 42 in the initial state of the catheter assembly 12a. The user can cause the guide wire 20 to protrude from the distal end opening 42 of the needle body 32 by gripping the handle 104 and moving the same toward the slit valve 98.

In the catheter assembly according to the present invention, the movement restriction unit may be configured to restrict the movement of the guide wire in the proximal end direction with respect to the needle body by contact of the guide wire with the needle body in the initial state of the catheter assembly.

The present invention is not limited to the above-described embodiments, and various configurations can be taken without departing from the gist of the present invention.

Embodiments of the present disclosure are summarized below.

According to one embodiment, a catheter assembly (12, 12a) includes a catheter member (16) including a hollow catheter shaft (24), a needle member (18, 18a) including a hollow needle body (32) inserted into a lumen (28) of the catheter shaft, and a guide wire (20) inserted into a lumen (36) of the needle body, in which a distal end opening (42) communicating with the lumen of the needle body is formed on a blade surface (40) of a distal end of the needle body, a light emission unit (66) that emits near-infrared light (L2) is provided on a distal end of the guide wire, and the light emission unit is located so as to overlap the distal end opening in a plan view in which the blade surface is seen in a direction orthogonal to an axis of the needle body in an initial state.

In the catheter assembly described above, the needle member may include a needle hub (34, 34a) provided on a proximal end of the needle body, and the catheter assembly may include a movement restriction unit (54, 54a) that restricts movement of the guide wire in a proximal end direction with respect to the needle body in the initial state.

In the catheter assembly described above, the distal end opening may include an opening center portion (44) located at a center portion in an axial direction of the needle body, and the light emission unit may be located in the proximal end direction from the opening center portion in the initial state.

In the catheter assembly described above, the distal end opening may include an opening proximal end (46) located on a proximal end in the axial direction of the needle body, and the light emission unit is located so as to overlap the opening proximal end in a plan view in the initial state.

In the catheter assembly described above, the light emission unit may include a fluorescent material that emits the near-infrared light when being irradiated with light.

The above-described embodiment discloses a blood vessel puncture system (10) including the catheter assembly described above, an irradiation unit (74) that irradiates a distal end of the catheter assembly with which a living body site (200) is punctured with light (L1) in a near-infrared region, a light reception unit (76) that receives reflected light reflected by the living body site and the catheter assembly out of the light and the near-infrared light emitted by the light emission unit to create a light reception image (82), and an image display unit (78) that displays the light reception image.

What is claimed is:

1. A catheter assembly comprising:
a catheter member comprising a hollow catheter shaft;
a needle member comprising a hollow needle body located in a lumen of the catheter shaft; and
a guide wire located in a lumen of the needle body; wherein:
a distal end opening communicating with the lumen of the needle body is formed on a blade surface of a distal end of the needle body;
a light emission unit configured to emit near-infrared light is located on a distal end of the guide wire; and
in a plan view in which the blade surface is viewed in a direction orthogonal to an axis of the needle body in an initial state, the light emission unit overlaps the distal end opening.

2. The catheter assembly according to claim 1, wherein:
the needle member comprises a needle hub located at a proximal end of the needle body; and
the catheter assembly comprises a movement restriction unit configured to restrict movement of the guide wire in a proximal end direction with respect to the needle body in the initial state.

3. The catheter assembly according to claim 1, wherein:
the distal end opening includes an opening center portion located at a center portion in an axial direction of the needle body; and
the light emission unit is located proximal of the opening center portion in the initial state.

4. The catheter assembly according to claim 3, wherein:
the distal end opening includes an opening proximal end located on a proximal end in the axial direction of the needle body; and
in the plan view in the initial state, the light emission unit overlaps the opening proximal end.

5. The catheter assembly according to claim 1, wherein:
the light emission unit comprises a fluorescent material that emits the near-infrared light when irradiated with light.

6. A blood vessel puncture system comprising:
the catheter assembly according to claim 1;
an irradiation unit configured to irradiate a distal end of the catheter assembly with which a living body site is punctured with light in a near-infrared region;
a light reception unit configured to receive reflected light reflected by the living body site and the catheter assembly out of the light and the near-infrared light emitted by the light emission unit to create a light reception image; and
an image display unit configured to display the light reception image.

7. A catheter assembly comprising:
a catheter member comprising a hollow catheter shaft;
a needle member comprising a hollow needle body located in a lumen of the catheter shaft; and
a guide wire located in a lumen of the needle body; wherein:
a distal end opening communicating with the lumen of the needle body is formed on a blade surface of a distal end of the needle body;
a phosphorescent material applied to a distal end of the guide wire; and
in a plan view in which the blade surface is viewed in a direction orthogonal to an axis of the needle body in an initial state, the phosphorescent material overlaps the distal end opening.

8. A method for determining whether a blade surface of a needle body is inserted in a blood vessel, the method comprising:
providing a catheter assembly comprising:
a catheter member comprising a hollow catheter shaft,
a needle member comprising a hollow needle body located in a lumen of the catheter shaft, and
a guide wire located in a lumen of the needle body, wherein:
a distal end opening communicating with the lumen of the needle body is formed on a blade surface of a distal end of the needle body;
a light emission unit configured to emit near-infrared light is located on a distal end of the guide wire, and
in a plan view in which the blade surface is viewed in a direction orthogonal to an axis of the needle body in an initial state, the light emission unit overlaps the distal end opening;
using an irradiation unit, irradiating a distal end of the catheter assembly with which a living body site is punctured with light in a near-infrared region;
using a light reception unit, receiving reflected light reflected by the living body site and the catheter assembly out of the light and the near-infrared light emitted by the light emission unit, and creating a light reception image; and
using a display unit, displaying the light reception image.
